(19) United States
(12) Patent Application Publication
(10) Pub. No.: US 2026/0144311 A1
(43) Pub. Date: May 28, 2026

(54) EXERCISE OR SPORTING GARMENT WITH SENSORS

(71) Applicant: Vada Digital Limited, Auckland (NZ)

(72) Inventor: Alan Christopher Nicholas, Auckland (NZ)

(21) Appl. No.: 19/299,704

(22) Filed: Aug. 14, 2025

(30) Foreign Application Priority Data
Nov. 26, 2024 (AU) 2024903899
Jun. 10, 2025 (AU) 2025902346

(51) Int. Cl. A41D 13/00; A41D 13/015; A41D 13/02; A61B 5/00

(52) U.S. Cl. CPC A41D 13/0002; A41D 13/015; A41D 13/02; A61B 5/6804; A41D 2600/10

(57) ABSTRACT

An exercise or sporting garment for a wearer comprising one or more sensors integrated into the garment and one or more protective zones. The or each sensor is configured to sense and send biometric or physiological data about the wearer to a portable computing device or a remote electronic device. The or each protective zone comprises a plurality of overlaid pads arranged to protect a region of the wearer's body and flex with the wearer's movement. The portable computing device or the remote electronic device is configured to analyse the biometric or physiological data received from at least one of the sensors.

### CROSS-REFERENCE TO RELATED APPLICATION(S)

This application claims priority under 35 U.S.C. § 119 (a) to Australian Patent Application No. 2024903899, filed Nov. 26, 2024; and Australian Patent Application No. 2025902346, filed Jun. 10, 2025, which are each hereby incorporated by reference in their entirety.

### FIELD OF THE INVENTION

This invention relates to an exercise or sporting garment with one or more integrated sensors to monitor the wearer's athletic performance, particularly while training for, playing, or participating in a contact sport or other strenuous physical activity, such as rugby.

### BACKGROUND

Data analytics in sports provides a multitude of benefits. Coaches and medical professionals can use data collected from sportspeople to monitor athletic performance and provide insights that help improve physical training, tactics, rehabilitation, and injury prevention. By tracking biometrics like speed, heart rate, and movement patterns, the athlete's physical performance can be optimised, and preventative measures can be taken when the data indicates a risk of injury, for example, due to overtraining or fatigue.

The landscape of contact sports is evolving with rising concerns around player safety, particularly due to increased injury risks. Rugby is the world's fastest-growing contact sport, with increasing numbers of people playing it, attending games, and watching it on television. The physical nature of the sport means that players have a significant risk of injury during games and training, including concussions and dislocated shoulders.

Traditional protective gear for contact sports, such as helmets or shoulder pads, focuses primarily on reducing impact but lacks integrated systems that simultaneously enhance performance and assist in recovery. Furthermore, current wearable technologies focus on fitness metrics without addressing the specific needs of contact sports athletes. For example, training vests with built-in heart rate sensors do nothing to physically prevent or reduce the risk of injury and store the gathered data for future analysis rather than provide real-time analysis.

It is an object of at least preferred embodiments of the present invention to provide an exercise or sporting garment that mitigates at least one of the abovementioned disadvantages and/or to at least provide the public with a useful alternative.

### SUMMARY OF THE INVENTION

According to a first aspect of the invention, there is provided an exercise or sporting garment for a wearer, the garment comprising:
a) one or more sensors integrated into the garment and configured to sense and send biometric or physiological data about the wearer to a portable computing device or a remote electronic device; and
b) one or more protective zones, the or each protective zone of the garment comprising a plurality of overlaid pads arranged to protect a region of the wearer's body and flex with the wearer's movement;

wherein the portable computing device or the remote electronic device is configured to analyse the biometric or physiological data received from at least one of the sensors.

According to a second aspect of the invention, there is provided an exercise or sporting garment for a wearer, the garment comprising:
a) a carrier arranged to removably carry a computing device;
b) one or more sensors integrated into the garment and configured to sense and send biometric or physiological data about the wearer to the computing device; and
c) one or more protective zones, the or each protective zone of the garment comprising a plurality of overlaid pads arranged to protect a region of the wearer's body and flex with the wearer's movement;

wherein the computing device is configured to analyse the biometric or physiological data received from at least one of the sensors and transmit the data or information derived at least in part from the analysis of the data to a remote electronic device.

In one embodiment, the computing device is configured to analyse the biometric or physiological data received from at least one of the sensors and transmit the data or information in real-time.

In one embodiment, the garment is configured to dynamically adjust one or more characteristics of the or each protective zone.

In one embodiment, the one or more characteristics comprise compression, rigidity, or thickness of the plurality of pads.

In one embodiment, the or each sensor is integrated into the one or more protective zones.

In one embodiment, the or each protective zone comprises a sensor interposed between an inner impact-absorbing pad and an outer impact-absorbing pad.

In one embodiment, the garment comprises a fabric layer, the inner impact-absorbing pad of the or each protective zone abutting the fabric layer, and the outer impact-absorbing pad of the or each protective zone being substantially thicker than the inner impact-absorbing pad.

In one embodiment, the or each protective zone comprises a rigid or semi-rigid protective layer covering or partially covering the outer impact-absorbing pad.

In one embodiment, the or each protective zone comprises an armour layer covering or partially covering the protective layer.

In one embodiment, the or each protective zone is arranged to protect a region of the wearer's body selected from the wearer's shoulder, chest, abdomen, flank, back, and thigh.

In one embodiment, the garment contains one or more electrical conduits, electrical connection networks, or data bus having a first end region operably connected to the carrier or the computing device and a second end region operably connected to the or at least one sensor.

In one embodiment, the carrier is a docking structure adapted to releasably receive the computing device.

In one embodiment, the or each sensor comprises a sensor selected from an ECG sensor, a PPG sensor, a blood oxygen saturation sensor, a blood glucose sensor, a blood lactate sensor, a GPS sensor, a g-force or cumulative workload sensor, a hydration sensor, a temperature sensor, and a VO₂ max sensor.

According to a third aspect of the invention, there is provided a computing device arranged to be carried in a carrier on an exercise or sporting garment for a wearer, the garment comprising:
a) one or more sensors integrated into the garment and configured to sense and send biometric or physiological data about the wearer to the computing device; and
b) one or more protective zones, the or each protective zone of the garment comprising a plurality of overlaid pads arranged to protect a region of the wearer's body and flex with the wearer's movement;

wherein a processing unit of the computing device is configured to analyse the biometric or physiological data received from at least one of the sensors and transmit the data or information derived at least in part from the analysis of the data to a remote electronic device.

In one embodiment, the processing unit is configured to transmit the biometric or physiological data or information derived at least in part from the analysis of the data to a remote electronic device in real time.

In one embodiment, the computing device is configured to generate predictive workload insights for the wearer at least in part from the biometric or physiological data and transmit this information to the remote electronic device.

In one embodiment, the computing device is configured to assess cumulative fatigue or injury risk for the wearer at least in part from the biometric or physiological data and transmit this information to the remote electronic device.

In one embodiment, the computing device is configured to identify anomalous impact patterns or physiological stress responses at least in part from the biometric or physiological data and transmit this information to the remote electronic device.

According to a fourth aspect of the invention, there is provided a method of analysing biometric or physiological data about a wearer of an exercise or sporting garment, the method using a processing unit of a computing device configured to implement the steps of:
a) receiving the data from one or more sensors integrated into the garment;
b) analysing the data to derive information at least in part from the analysis of the data; and
c) transmitting the data or the information derived from the data to a remote electronic device;

wherein the garment comprises a carrier arranged to removably carry the computing device and one or more protective zones, the or each protective zone comprising a plurality of overlaid pads arranged to protect a region of the wearer's body and flex with the wearer's movement.

In one embodiment, the method further comprises the step of sending an alert to the remote electronic device that the wearer is at an increased risk of injury or fatigue based at least in part on the analysis of the biometric or physiological data.

In one embodiment, the biometric or physiological data comprises any one or more of the following: cardiac data indicative or representative of the health or condition of the wearer's heart; blood oxygen data indicative or representative of the heart rate or blood oxygen level of the wearer; blood volume data indicative or representative of the blood oxygen level of the wearer; blood glucose data indicative or representative of the blood glucose level of the wearer; blood lactate data indicative or representative of the blood lactate concentration of the wearer; GPS data indicative or representative of the location or movement of the wearer; g-force or impact intensity data indicative or representative of the acceleration force on the wearer's body; hydration data indicative or representative of the hydration level or water content of the wearer's body; temperature data indicative or representative of the core temperature of the wearer's body; and VO₂ max data indicative or representative of the VO₂ max level of the wearer's body.

According to a fifth aspect of the invention, there is provided an electronically-implemented method comprising software code or coded instructions that are executable or implemented by a computer processor, or controller to carry out the method of the fourth aspect of the invention.

The term ‘comprising’ as used in this specification and claims means ‘consisting at least in part of’. When interpreting statements in this specification and claims that include the term ‘comprising’, other features can also be present besides the features prefaced by this term in each statement. Related terms such as ‘comprise’ and ‘comprised’ should be interpreted similarly.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended, and all possible combinations of numerical values between the lowest value and the highest value enumerated are to be considered to be expressly stated in this application similarly.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

As used herein, the term ‘(s)’ following a noun means that noun's plural and/or singular form.

As used herein, the term ‘and/or’ means ‘and’ or ‘or’, or where the context allows both. The invention consists of the foregoing and also envisages constructions, of which the following gives examples only.

### BRIEF DESCRIPTION OF THE DRAWINGS

The present invention will now be described by way of example only and with reference to the accompanying drawings in which:

FIG. 1 shows an schematic diagram of an exercise monitoring system according to an embodiment of the invention.

FIG. 2 shows a front perspective view of an exercise or sporting garment for use in the exercise monitoring system of FIG. 1.

FIG. 3 shows a rear perspective view of the garment shown in FIG. 2.

FIG. 4 shows a front perspective view of sensors integrated into protective zones of the garment of FIG. 2.

FIG. 5 shows a rear perspective view of a computing device and sensors integrated into protective zones of the garment of FIG. 2.

FIG. 6 shows a front perspective view of a first embodiment of a computing device for use with the garment of FIG. 2.

FIG. 7 shows a side perspective view of a second embodiment of a computing device for use with the garment of FIG. 2.

FIG. 8 shows a side perspective view of the second embodiment of the computing device of FIG. 7 and a docking structure.

FIG. 9 shows a cross-sectional side view of a portion of the second embodiment of the computing device of FIG. 7.

FIG. 10 shows a plan perspective view of a first embodiment of a protective zone of the garment of FIG. 2.

FIG. 11 shows an exploded view of the first embodiment of the protective zone.

FIG. 12 shows a cross-sectional side view of the first embodiment of the protective zone.

FIG. 13 shows a side perspective view of a second embodiment of a protective zone of the garment of FIG. 2.

FIG. 14 shows an exploded view of the second embodiment of the protective zone.

FIG. 15 shows a cross-sectional side view of the second embodiment of the protective zone.

FIG. 16 shows a side perspective view of a third embodiment of a protective zone of the garment of FIG. 2.

FIG. 17 shows an exploded view of the third embodiment of the protective zone.

FIG. 18 shows a cross-sectional side view of the third embodiment of the protective zone.

FIG. 19 shows a side perspective view of a fourth embodiment of a protective zone of the garment of FIG. 2.

FIG. 20 shows an exploded view of the fourth embodiment of the protective zone.

FIG. 21 shows a cross-sectional side view of the fourth embodiment of the protective zone.

FIG. 22 shows a flowchart of a process for wirelessly transmitting data from the sensors in the garment of FIG. 2 to a remote electronic device.

### DETAILED DESCRIPTION OF THE PREFERRED EMBODIMENTS

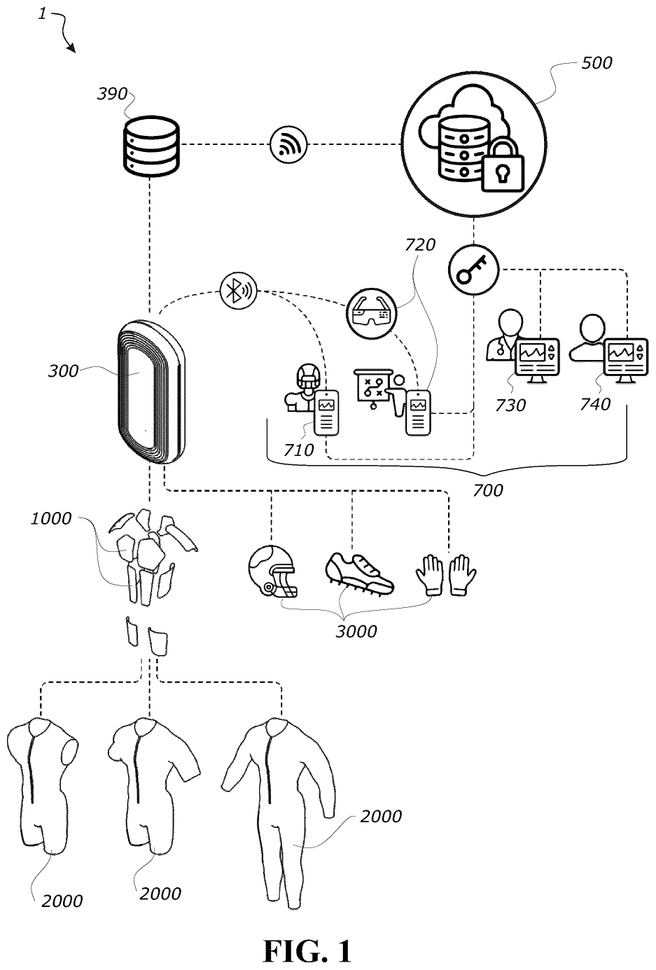

FIG. 1 illustrates a schematic diagram of an exercise monitoring system 1 according to an embodiment of the invention. The exercise monitoring system 1 includes an exercise or sporting garment 100 for a wearer, such as a professional athlete or soldier.

In the exemplified embodiment, the garment 100 comprises a customised bodysuit made of a fabric layer 2000 covering the wearer's torso and portions of the wearer's arms and legs, to which a carrier 4000 is fixed or fixable to carry a removable computing device 300. The garment 100 also comprises one or more sensors integrated into the garment and configured to sense and send biometric or physiological data about the wearer to the computing device 300. One or more sensors may also be integrated into auxiliary garments or wearables 3000, such as, helmets, shoes, or gloves. The garment 100 further comprises one or more protective zones 1000, the or each protective zone comprising a plurality of overlaid or overlapping pads arranged to protect a region of the wearer's body and flex with the wearer's movement. In the exemplified embodiment, the sensor(s) are integrated into the protective zone(s) 1000.

The computing device 300 is configured to analyse the biometric or physiological data received from at least one of the sensors. The computing device 300 has an internal memory 390, such as edge memory, for storing the biometric or physiological data and/or information derived at least in part from the analysis of the data. The data and/or information can be wirelessly sent from the internal memory 390 to an encrypted cloud-based database 500, where at least a portion of the data and/or information can be securely accessed on one or more remote electronic devices 700, such as a personal computer, laptop computer, tablet computer, or mobile computer (such as a smartphone, smartwatch or smartglasses) or any other suitable electronic device by one or more people.

All data transmissions from the computing device 300 to the remote electronic device(s) are protected using AES-256 encryption and TLS 1.3 for secure wireless communication. The exercise monitoring system 1 enforces multi-factor authentication (MFA), role-based access controls (RBAC), and end-to-end encryption for both data at rest and in motion. The exercise monitoring system 1 also includes a blockchain-based data management architecture that ensures secure, decentralised ownership of all wearer-generated biometric and physiological data. Each data record is cryptographically stored on a private blockchain, enabling immutable logging and user-directed access permissions. Wearers retain full control over their personal data through smart contracts, allowing selective authorisation of data access by teams, medical personnel, or third parties. This ensures data portability and compliance with the Health Insurance Portability and Accountability Act (HIPAA), General Data Protection Regulation (GDPR), and International Traffic in Arms Regulations (ITAR).

The remote electronic device(s) 700 may comprise the wearer's personal mobile computer 710 which can be configured to receive real-time Bluetooth or 5G data streams from the computing device 300. Additionally, a coach's remote electronic device 720, typically a tablet or laptop, may display real-time performance analytics, cumulative impact data, and injury risk assessments for the wearer based on the outputs of the computing device 300. A remote electronic device 730 may allow sports medics, military healthcare personnel, or recovery specialists to review an athlete or soldier's physiological and impact profiles in real time for personalised recovery programming and injury prevention strategies. A remote electronic device 740 may also provide access to data and/or information to authorised external entities, such as, insurance providers, athlete agents, or military oversight bodies.

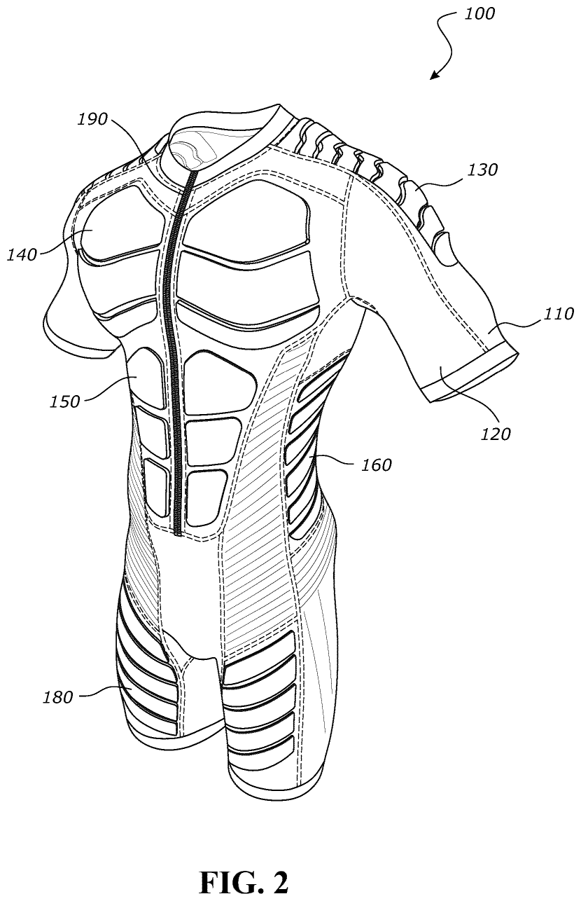

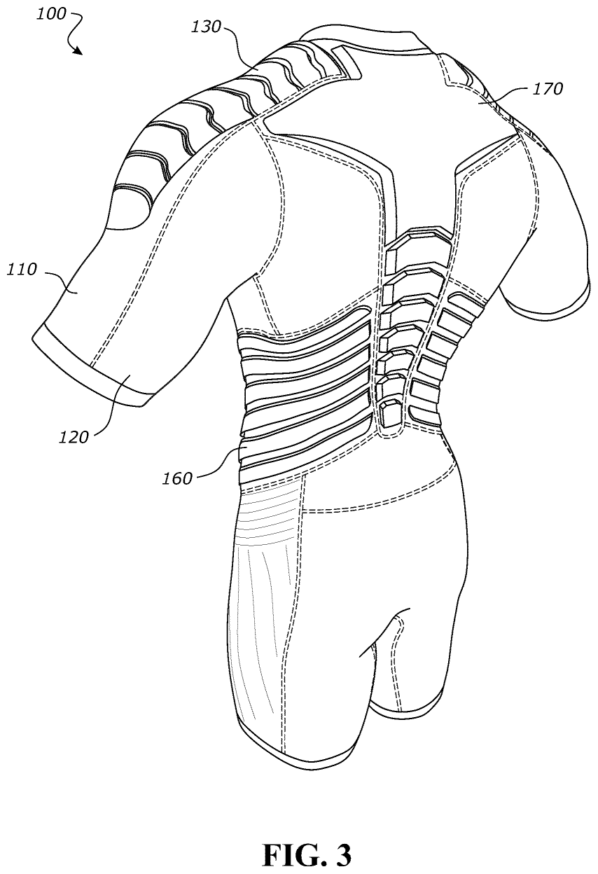

As shown in FIGS. 2 and 3, the garment 100 of the system comprises a close-fitting, one-piece bodysuit for a wearer that covers the wearer's torso and portions of the wearer's arms and legs. However, the garment 100 could be any other suitable exercise or sporting garment for a wearer. The garment 100 is intended to be worn by someone who plays rugby or another physically demanding sport (such as American football, ice hockey, lacrosse, mountain biking, or motorsport) during training or a game, or someone who has a physically demanding job (such as military personnel) during training or the performance of their job, such as during military operations. The garment 100 is tailored to the specific needs of contact sports or other physically challenging activities, allowing the wearer to perform at their best while receiving the necessary protection from impacts. The garment 100 can be customised based on an individual wearer's gender and specific needs. It will be appreciated that the garment 100 could also be worn, with or without modification, by non-contact sportspeople or athletes.

The garment 100 is primarily made from sections of a stretchable fabric layer 2000, which have been sewn together along the seams shown in broken lines. Examples of two of these fabric sections that, when sewn together, form a sleeve of the garment 100 are shown in FIGS. 2 and 3 as 110 and 120. Each fabric section 110, 120 is made of a high-performance material, such as a moisture-wicking fabric or breathable mesh for the wearer's comfort and temperature regulation, for example, one or more elastomers, textiles, and composite fabrics with high tensile strength and elasticity, e.g., reinforced polymer threads and nanofiber composites to provide durability and adaptability.

The garment's construction also includes several protective zones 1000, each zone comprising a plurality of overlaid, flexible, impact-absorbing foam pads 1200, 1300 and durable, flexible composite materials in specific regions to protect the wearer's shoulders 130, chest 140, abdomen 150, flanks 160, back (e.g., between the shoulder blades and/or over the spine 170), and thighs 180. These regions are where rugby players and other contact sport athletes receive the most physical impact, for example, during a tackle from an opposing team player or in a ruck or maul. The foam pads 1200, 1300 in each of these protective zones 1000 are spaced apart so as to flex with the wearer's movement, for example, the pads in the shoulder regions 130 should flex inwardly to narrow or close the spaces between the pads when the wearer lifts their arms above their head. The pads and the spaces between them are designed to reduce the risk of soft tissue damage and joint injuries while preserving mobility.

Each protective zone 1000 can include a dynamic compression system integrated into the fabric layer 2000 of the garment 100 to provide additional stability to joints and muscles and improve blood flow during exercise, especially for wearers recovering from injuries or prone to repetitive stress injuries. Each protective zone 1000 can also include a recovery system, including adjustable hot and cold regions to aid in post-exercise recovery. Each protective zone 1000 may include at least one adaptive response system configured to dynamically adjust characteristics of compression, rigidity, or padding thickness in real time based on the wearer's biometric or physiological data, such as impact intensity and location, processed by an onboard artificial intelligence (AI) engine, such as a reasoning engine, in the computing device 300. The adaptive response system can be pre-programmed for sport-specific contact thresholds. A zipper or zip fastener 190 extending from the collar to approximately the wearer's navel is located at the front of the garment 100, but could alternatively be located at the back. It will be appreciated that the garment 100 could be in a sports team's colours and feature one or more names or logos, such as the names or logos of the garment manufacturer, sports team, or team sponsor. For a military application, the garment 100 could be in one or more colours that provide camouflage for the wearer.

The close-fitting garment 100 may be custom-fit for an individual wearer, achieved using three-dimensional (3D) body scanning technology. Wearers can undergo a detailed 3D body scan identifying areas prone to injury, muscle imbalances, and anatomical differences. This information is used to create a garment 100 tailored to the wearer's body type, injury history, and performance needs, providing targeted compression and protection where it is needed most.

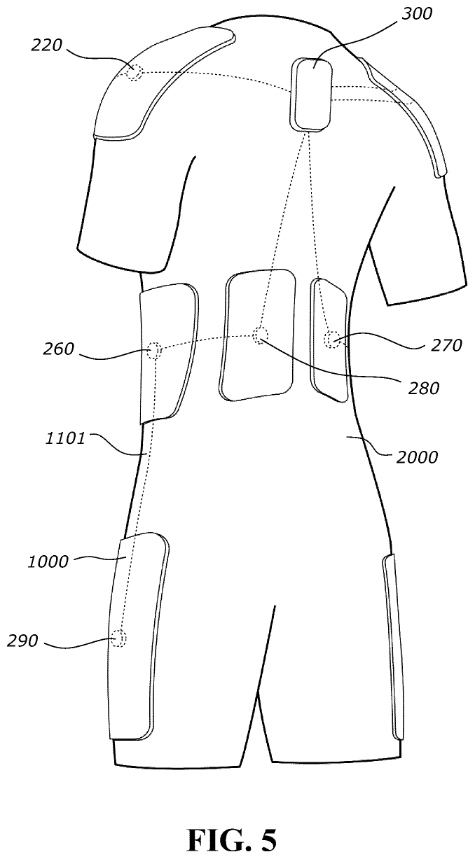

In one embodiment, the garment 100 includes a carrier 4000, such as a pocket, docking structure, holster, or other suitable carrier, to removably carry a portable or removable computing device 300. FIG. 5 shows that the computing device 300, when carried inside the carrier (not shown), is held against the wearer's upper back between the shoulder blades. However, it will be appreciated that the carrier 4000 could be located at other locations in the garment 100 and that, rather than be removable, the computing device 300 could be integrated into the garment 100. When the carrier 4000 is a pocket, the wearer may open the pocket to insert the computing device 300 into or remove the computing device from the garment 100 and close the pocket using at least one fastener selected from any one or more of the following: at least one zipper, at least one button, at least one hook, or any other suitable fastener.

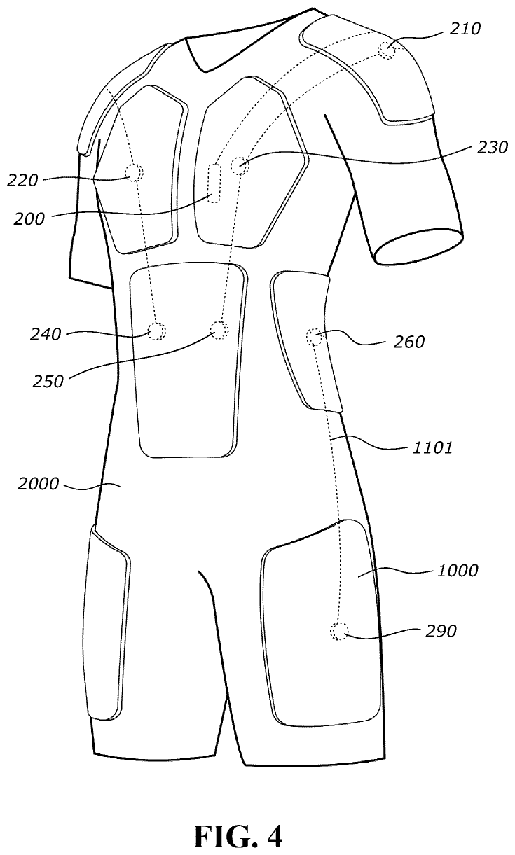

As shown in FIGS. 4 and 5, the garment 100 contains a plurality of spaced-apart miniaturised sensors 200-290 strategically integrated, embedded or placed within the garment's protective zones 1000 to protect the sensors during impact. The sensors 200-290 may be flexible sensors capable of a range of movement. Each sensor 200-290 monitors at least one physiological metric or biometric of the wearer. For example, sensor 200 shown in FIG. 4 is positioned close to the wearer's heart to monitor heart rate variability. The other sensors 210-290 are each positioned at various spaced-apart locations in the garment 100 to monitor biometrics such as muscle activation (via electromyography), joint range of motion, GPS for tracking player movements, impact forces experienced during tackles or collisions, and sweat and hydration levels through smart fabric technology. In one embodiment, each sensor 200-290 communicates, wired or wirelessly, with the computing device 300 for real-time feedback and analysis.

Traditionally, there can be a substantial delay between an athlete performing an activity during training or a game and analysing the athlete's performance after the training or in a post-game review, which increases the risk of injury and reduces the timeliness of coaching to optimise performance. In contrast, the garment 100 allows coaches, medical professionals, and wearers to access performance data during games or practice, enabling quick adjustments to strategy or technique, such as on-field tactical or medical decisions. In one embodiment, each sensor 200-290 communicates directly with a remote electronic device 700 via a wireless connection.

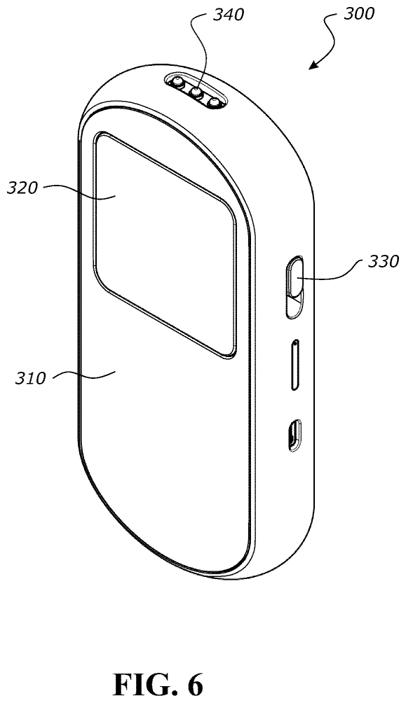

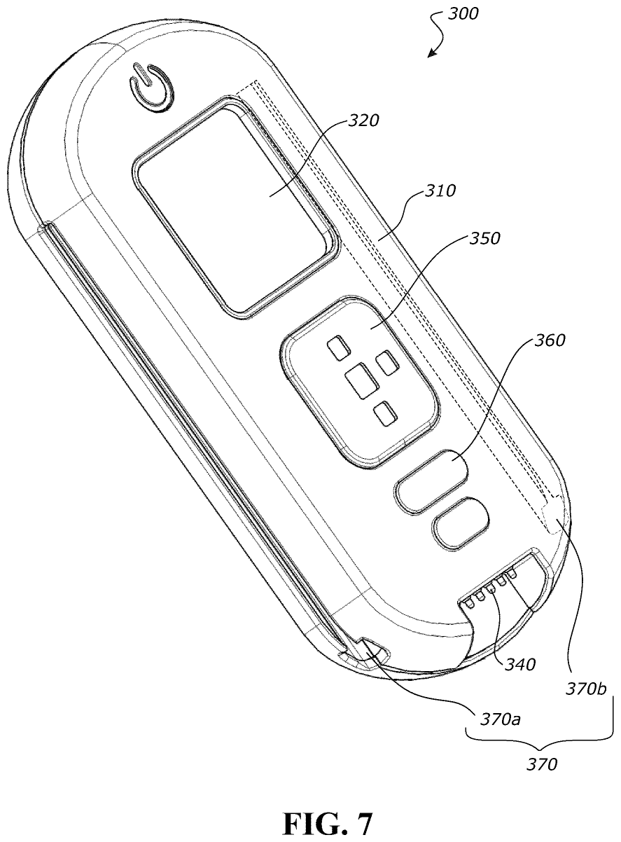

FIGS. 6 and 7 show two embodiments of the computing device 300. The computing device 300 may be a lightweight, compact, handheld device sized to fit inside or be received by a carrier 4000 or otherwise be integrated into the garment 100 without negatively impacting the wearer's movement or athletic performance. The computing device 300 may be elongate with a length of about 100 mm, a height of about 50 mm, and a width of about 15 mm. The computing device 300 should preferably weigh less than 200 g to 700 g. As shown in FIGS. 6 and 7, the computing device 300 comprises a housing 310 encasing a processing unit 380, a memory 390, at least one communication module, and a power source. The exterior surface of the housing 310 should be shaped so as not to have any sharp corners, such as the stadium or pill shape shown in FIGS. 6 and 7. The housing 310 is preferably made of rugged, waterproof materials (e.g., IP67 or higher rating) for durability in extreme sporting conditions, for example, a suitable plastic, such as thermoplastic polyurethane (TPU).

The computing device 300 may have a user interface comprising a display or touch screen 320, such as an E Ink (electronic ink) screen, integrated into the housing 310 and, optionally, one or more buttons 330 to receive the user's input, such as powering the device on or off or pairing the computing device with other devices. The display or touch screen 320 can display personalised information to the user, such as the wearer's name and/or uniform or identification number, biometric readings, alerts, and the current status or operating mode of the device. The computing device 300 also has a connector 340, such as one or more spring-mounted pins, configured to operably connect the computing device to an end region 1102 of at least one electrical conduit 1101 in the garment 100. As shown in FIG. 5, the other end region 1103 of the electrical conduit(s) 1101 is connected to at least one of the sensors 200-290 in the garment 100. The connector 340 facilitates secure data and/or power transmission between the computing device 300 and the sensors 200-290. It will be appreciated that, instead of each sensor 200-290 having its own direct electrical connection to the computing device 300 via an electrical conduit(s), the sensors could share a serial data bus or other electrical network connection.

The computing device 300 may contain one or more sensors embedded in the housing 310 of the device. For example, in the embodiment of the computing device 300 shown in FIG. 7, the computing device contains an electrocardiography (ECG) sensor 350 configured to monitor the wearer's heart rate and heart rate variability (HRV), and a hydration sensor 360 configured to estimate fluid levels within the wearer's body by measuring the skin's impedance. However, it will be appreciated that any of the sensors 200-290 located in the garment 100 could be located in the computing device 300.

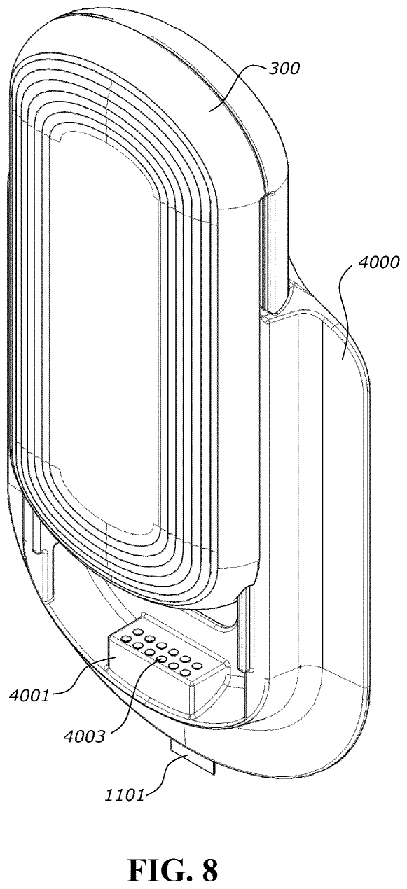

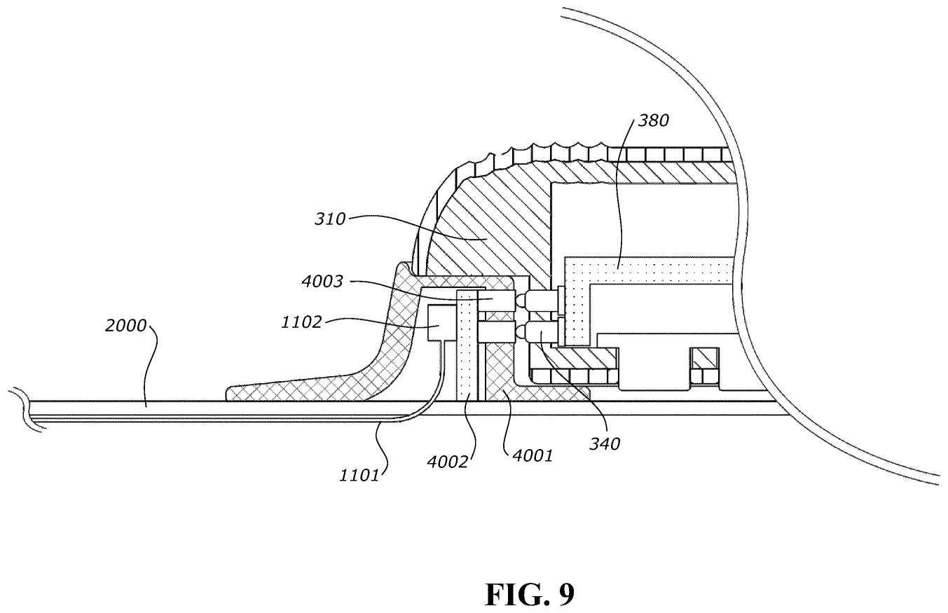

The computing device 300 may contain one or more coupling elements 370 to releasably couple the device to the garment 100. For example, in the embodiment of the computing device shown in FIGS. 7 to 9, the computing device contains two spaced apart slots 370a, 370b formed opposing surfaces of the housing 310, each slot configured to slidably and releasably receive a portion of a carrier 4000 in the form of a docking structure fixed or fixable to the fabric layer 2000 of the garment 100. The carrier 4000 is adapted to retain the computing device 300 against the garment 100 and may be made of any suitable rigid or semi-rigid material. FIG. 9 shows a portion of the computing device 300 when coupled to the carrier 4000 on the garment 100. As shown, while coupled, an end region 1102 of at least one electrical conduit 1101 is operably connected to the connector 340 of the computing device 300 via a connector interface 4002 and an electrical contact 4003 in a protruding region 4001 of the carrier 4000.

The processing unit 380 of the computing device 300 comprises a main processor for processing the data received from the sensors 200-290 and at least one graphics processing unit (GPU) for processing digital images for transmission using the communication module(s). The main processor should be capable of handling high-frequency data streams from the sensors 200-290 with minimal delay. The main processor may be configured for edge computing, allowing for multi-threaded processing of the data streams. The computing device 300 may include an edge computing module configured to process large volumes of sensor data locally, reducing reliance on external servers and enhancing response time for real-time workload management. The main processor is preferably energy-efficient, such as an ARM Cortex-A78 or an x86-based Intel Atom processor. The processing unit 380 includes at least one AI-optimised GPU configured with on-device machine-learning applications for real-time analysis of biomechanical and physiological metrics from the sensor data. At least one GPU may be configured for parallel processing, enabling fast interpretation of large datasets, such as movement and force patterns, without offloading to cloud servers. An example of a device with a suitable GPU is the NVIDIA Jetson Nano. However, any other suitable AI-optimised GPU could be used.

The computing device's onboard memory 390 is accessible by the processing unit 380, which stores software applications and data received from the sensors 200-290. Between 8 GB to 16 GB of random-access memory (RAM), such as low-power double data rate (LPDDR) RAM, can be used to operate the machine learning applications, ensuring fast processing of real-time data streams and support for on-device machine learning tasks. A solid-state drive (SSD), such as the 256 GB NVMe SSD, can be used to store high-resolution data locally for post-session analysis and logs of training sessions.

The communication module(s) of the computing device 300 are configured to communicate data with the sensors 200-290 affixed to the garment 100 and/or one or more external devices or servers over a link or network, whether wired, wireless or both. In one embodiment, the communication module(s) are configured to have a transmitter, receiver, and/or transceiver to enable the computing device 300 to send and receive data wirelessly to or from the sensors 200-290 and other external devices or servers, such as a remote electronic device 700 with a display for a coach, medical professional, or other user to view. The remote electronic device 700 could comprise a personal computer, laptop computer, tablet computer, mobile computer (such as a smartphone, smartwatch, or smartglasses), or any other suitable device. The remote electronic device 700 may comprise a handheld device that allows the coach, medical professional, or other user to view the data from the sidelines of a sporting venue or fitness facility during a game or practice session. In one embodiment, the remote electronic device 700 comprises a device configured to be worn by the coach, medical professional, or other user, such as smartglasses, augmented reality (AR) glasses, or heads-up display (HUD) glasses, to visualise the data. In another embodiment, the remote electronic device 700 comprises a device, such as a smartwatch, configured to be worn by the wearer and provide haptic feedback or visual notifications to signal injury risk or fatigue thresholds directly to the wearer.

The communication module(s) could comprise Wi-Fi, Bluetooth, infrared (IR), radio frequency (RF), and/or cellular modules. For example, Wi-Fi 6 can be used for high-speed data transfer and minimal latency during data syncing, Bluetooth 5.0+ for low-energy continuous connection to companion devices or adaptive trainer dashboards, and 5G cellular for remote connectivity when Wi-Fi is unavailable to ensure real-time data access in diverse environments. Any suitable Wi-Fi module may be used, including but not limited to the Qualcomm QCA9377 Wi-Fi 6 chip. Any suitable Bluetooth module may be used, including but not limited to the Nordic Semiconductor nRF52840 with Bluetooth 5.2 support. Any suitable 4G or 5G cellular module may be used, including but not limited to the Quectel EC25 module. Alternatively, the communication module(s) may be configured to transmit and receive data over a wired connection with the sensors 200-290 and/or the remote electronic device 700, such as an IP68 USB-C or other suitable connection for data transfer and, optionally, charging. The computing device may have standardised ports to receive direct inputs from one or more connected sensors affixed to the garment 100.

The power source of the computing device 300 can be a battery. The battery is preferably rechargeable, such as a 3,000 mAh to 5,000 mAh lithium-ion or lithium polymer battery, allowing up to 12 hours of continuous operation under typical use. An AI-based power management application can extend the battery life during high-demand data processing sessions. The power source can be configured to quickly charge the battery via a USB-C cable, providing a full recharge within one to two hours. Alternatively, the battery can be charged wirelessly. The amount of charge on the battery can be shown to the user on the display or touch screen 320, for example, as a graphical image.

The garment 100 contains one or more sensors 200-290 integrated (e.g., embedded or sewn) into the fabric layer 2000 or other material of the garment at different locations, such as a fabric section or protective zone 1000. Each sensor 200-290 is selected from the group consisting of an electrocardiography (ECG) sensor, a photoplethysmography (PPG) sensor, a blood oxygen saturation sensor, a blood glucose sensor, a blood lactate sensor, a global positioning system (GPS) sensor, a g-force (gravitational force equivalent) or cumulative workload sensor, a hydration sensor, a temperature sensor, and a VO₂ max sensor. Each of these ten sensor types is described in detail below.

Combining data from multiple sensors 200-290 in real time provides a holistic view of the wearer's athletic performance and health metrics. Where the garment 100 contains a plurality of sensors 200-290, the sensors may be spaced apart from each other in the garment 100. Preferably, the garment 100 contains any two or more of the above sensors, more preferably any three or more of the above sensors, still more preferably any four or more of the above sensors, still more preferably any five or more of the above sensors, still more preferably any six or more of the above sensors, still more preferably any seven or more of the above sensors, still more preferably any eight or more of the above sensors, still more preferably any nine or more of the above sensors, and most preferably at least one of all 10 of the above sensors.

The processing unit 380 of the computing device 300 may be configured to send a real-time or immediate alert to the remote electronic device 700 that the wearer is at an increased risk of injury or fatigue based at least in part on the analysis of the biometric or physiological data sent to the computing device by the sensors 200-290. In particular, the processing unit 380 may be configured to send real-time fatigue alerts when a value in the biometric or physiological data is continuously above or below a predetermined threshold value for a set period. The processing unit 390 may compare the biometric or physiological data to historical data stored in memory 390 for AI-driven predictive training insights.

### ECG Sensor

The ECG sensor is configured to measure electrical impulses at a location on the wearer's skin, i.e., skin-surface voltage differences. These electrical impulses are caused by electrical signals originating in the sinoatrial node of the wearer's heart, which travel through the atria and ventricles and coordinate contractions. The electrical impulses can provide insights into the wearer's heart rate, rhythm, and overall cardiac health. Any suitable ECG sensor may be used, including but not limited to the MyoWare Muscle Sensor.

The processing unit 380 of the computing device 300 is configured to either receive cardiac data from the ECG sensor or determine cardiac data indicative or representative of the health or condition of the wearer's heart. The cardiac data may comprise the P wave (corresponding to atrial contraction), QRS complex (corresponding to ventricular depolarisation), and T wave (corresponding to ventricular repolarisation) components of an electrocardiogram. A GPU in the processing unit 380 can generate an electrocardiogram or ECG signal from the cardiac data, e.g., for transmission to a remote electronic device 700 for display to a coach, medical professional, or other user.

The processing unit 380 can be configured to analyse the cardiac data to diagnose heart irregularities, track heart rate variability (HRV) to determine the wearer's stress and fatigue and monitor cardiac responses during physical activity. A consistently elevated heart rate above baseline, especially during lower-intensity training, can indicate fatigue. Similarly, low HRV is also often linked to fatigue and slower recovery times. Abnormal increases in heart rate coupled with low HRV are, together, strong indicators of stress or overtraining, which are linked to increased injury risk. The cardiac data or information from its analysis can be transmitted, for example, to the remote electronic device 700. For example, an alert that the wearer is at an increased risk of injury or fatigue can be sent if their heart rate is continuously above a predetermined threshold value for a set period and/or their HRV is continuously below a predetermined threshold value for a set period.

### PPG Sensor

The PPG sensor is an optical sensor configured to measure blood volume changes at a location on the wearer's body by using light to penetrate the skin and detect blood flow variations in capillaries, indirectly gauging the wearer's heart rate and blood oxygen saturation. Blood flow varies with each heartbeat, affecting how much light is absorbed and reflected by blood in the skin. PPG sensors exploit this relationship to monitor blood volume changes, providing data on heart rate and blood oxygen levels. Any suitable PPG sensor may be used.

The processing unit 380 of the computing device 300 is configured to either receive blood volume data from the PPG sensor or determine blood volume data indicative or representative of the heart rate or blood oxygen level of the wearer. The processing unit 380 can be configured to analyse the blood volume data to continuously monitor the wearer's heart rate, blood oxygen saturation (SpO₂) levels, and heart rate variability (HRV) and assess workout intensity. The blood volume data or information from its analysis can be transmitted, for example, to the remote electronic device 700. Like the ECG sensor, an alert that the wearer is at an increased risk of injury or fatigue can be sent if their heart rate is continuously above a predetermined threshold value for a set period and their HRV is continuously below a predetermined threshold value for a set period.

### Blood Oxygen Saturation Sensor

The blood oxygen saturation sensor or pulse oximeter is an optical sensor configured to measure the amount of hemoglobin in the wearer's blood to determine blood oxygen saturation (SpO₂) levels. Like the PPG sensor, the blood oxygen saturation sensor uses light to penetrate the skin and measures the amount of absorbed light. Blood oxygen saturation is a critical indicator of respiratory efficiency and endurance. Oxygenated hemoglobin absorbs infrared light, while deoxygenated hemoglobin absorbs more red light. By measuring the ratio of red to infrared light absorbed by the wearer's blood, the blood oxygen saturation sensor can assess blood oxygen levels in real-time. Any suitable blood oxygen saturation sensor may be used, including but not limited to the Maxim MAX30101 or Texas Instruments AFE4700 pulse oximeters.

The processing unit 380 of the computing device 300 is configured to either receive blood oxygen data from the blood oxygen saturation sensor or determine blood oxygen data indicative or representative of the blood oxygen level of the wearer. The processing unit 380 can be configured to track blood oxygen saturation levels for monitoring the wearer's endurance, which is particularly useful in altitude training and assessing respiratory efficiency in athletes. A decrease in blood oxygen saturation, particularly during periods of high physical exertion, may indicate fatigue. The blood oxygen data or information from its analysis can be transmitted, for example, to the remote electronic device 700. For example, an alert that the wearer is at an increased risk of injury or fatigue can be sent if their blood oxygen saturation level is continuously below a predetermined threshold value for a set period.

### Blood Glucose Sensor

The blood glucose sensor is an optical sensor configured to measure the amount of glucose in the wearer's blood in a minimally invasive or non-invasive manner. Glucose is the body's main energy source, and maintaining stable levels is crucial for sustained energy, especially during prolonged physical exertion. Traditionally, monitoring blood glucose levels involves taking blood samples, but minimally invasive or non-invasive sensors use infrared light or other optical techniques to estimate glucose levels. Any suitable blood glucose sensor may be used, including but not limited to the Rockley Photonics Bioptx wristband sensor.

The processing unit 380 of the computing device 300 is configured to either receive blood glucose data from the blood glucose sensor or determine blood glucose data indicative or representative of the wearer's blood glucose level. The processing unit 380 can be configured to analyse or monitor the blood glucose data to detect hypoglycemia in wearers needing glucose management (e.g., diabetics) and for tracking energy availability during extended physical activities. Low blood glucose levels can indicate the wearer has reduced energy, potentially increasing the risk of accidents or injuries during intense physical activity. The blood glucose data or information from its analysis can be transmitted, for example, to the remote electronic device 700. For example, an alert that the wearer is at an increased risk of injury or fatigue due to low blood glucose can be sent if their blood glucose level is continuously below a predetermined threshold value for a set period.

### Blood Lactate Sensor

The blood lactate sensor is configured to measure the lactate concentration in the wearer's blood, which increases due to intense anaerobic exercise activity and can indicate fatigue. During high-intensity anaerobic activities, the body produces lactate as a byproduct of energy production, leading to muscle fatigue. Measuring lactate levels can reveal the wearer's anaerobic threshold, helping manage intensity and recovery. Any suitable blood lactate sensor may be used, including but not limited to a non-invasive sensor that measures the lactate in the wearer's sweat.

The processing unit 380 of the computing device 300 is configured to either receive blood lactate data from the blood lactate sensor or determine blood lactate data indicative or representative of the wearer's blood lactate concentration. The processing unit 380 can be configured to analyse or monitor the blood lactate data to provide insights into the wearer's anaerobic threshold, muscle fatigue, and endurance potential, which are valuable in real-time fatigue and recovery analysis. The blood lactate data or information from its analysis can be transmitted, for example, to the remote electronic device 700. For example, an alert that the wearer is at an increased risk of injury or fatigue can be sent if their blood lactate concentration is continuously above a predetermined threshold value for a set period.

### GPS Sensor

The GPS sensor is configured to track the wearer's location and movement using a satellite network that sends signals to a receiver in the sensor. The wearer's exact location, speed, and direction of movement can be determined by calculating the time taken for signals from multiple satellites to reach the receiver. Multiple GPS sensors, for example, one GPS sensor in each protective zone, can provide positional tracking, mapping the wearer's movement patterns and impact zones for sport-specific biomechanical analysis. Any suitable GPS sensor may be used, including but not limited to the u-blox NEO-M9N module.

The processing unit 380 of the computing device 300 is configured to either receive GPS data from the GPS sensor or determine GPS data indicative or representative of the location or movement of the wearer. GPS data may comprise the wearer's distance, speed, and positional data during activities. The processing unit 380 can be configured to analyse the GPS data to map the wearer's movement patterns and assess workload, such as a workload index tracking accumulated impact loads over training sessions or games, recovery cycles, and biometric trends to optimise training intensity and minimise overuse injuries. The GPS data or information from its analysis can be transmitted, for example, to the remote electronic device 700. For example, an alert that the wearer is at an increased risk of injury or fatigue can be sent if their speed or distance moved is continuously below a predetermined threshold value for a set period. The alert may comprise a real-time recommendation or prompt to substitute or replace the wearer, adjust training intensity, or provide medical treatment or fluids to the wearer. The alert may be personalised to the wearer's role or risk threshold.

### G-Force or Cumulative Workload Sensor

The g-force or cumulative workload sensor comprises an accelerometer and optional gyroscope configured to measure acceleration forces (g-forces) exerted on the wearer's body during movement. When the wearer's body accelerates or decelerates, the g-forces exerted on the body can be measured by the accelerometer and optional gyroscope of the sensor, capturing the wearer's intensity, rotation, and movement patterns. These measurements are crucial in understanding biomechanical stresses, especially in high-impact sports like rugby. High-impact sports produce repetitive forces that can lead to injuries if not managed. The sensor's accelerometer and gyroscope measure the direction and magnitude of forces, providing insights into cumulative strain on the wearer's body, risk factors for injuries from repeated impacts or bad technique, and tactical insights such as a defensive mismatch or declining effort. Any suitable g-force sensor may be used, including but not limited to the Analog Devices ADXL372 accelerometer or the Bosch BNO055 IMU chip combining an accelerometer and gyroscope. The g-force sensor can differentiate between direct impact forces, rotational forces, and repetitive sub-concussive impacts.

The processing unit 380 of the computing device 300 is configured to either receive g-force or impact intensity data from the g-force sensor or determine g-force or impact intensity data indicative or representative of the acceleration forces on the wearer's body. The g-force or impact intensity data may comprise the number and severity of impacts over a set period, contributing to insights into fatigue, injury risk, rehabilitation, and workload management. The processing unit 380 can be configured to analyse the g-force or impact intensity data to track physical impacts, the intensity of contacts, and the cumulative workload on the wearer's body, which is useful for monitoring collision forces in contact sports and calculating biomechanical stresses. High cumulative impact levels over time or repeatedly high spikes in acceleration forces indicate a higher risk of impact-related injuries, especially in contact sports. Additionally, sudden changes in velocity or direction can contribute to musculoskeletal injuries, especially when a wearer is fatigued. The g-force or impact intensity data or information from its analysis can be transmitted, for example, to the remote electronic device 700. For example, an alert that the wearer is at an increased risk of injury or fatigue can be sent if the acceleration forces exerted on the wearer are cumulatively or repeatedly above a predetermined threshold value for a set period.

### Hydration Sensor

The hydration sensor comprises at least one electrode configured to send a small electrical current through the wearer's skin and a detector configured to measure the skin's impedance to estimate fluid levels within the body. Hydration is essential for thermoregulation, electrolyte balance, and optimal muscle function. Using bioimpedance analysis (BIA), a small electrical current can be sent through the body, and since water conducts electricity well, the body's total water content can be estimated from the skin's impedance, which fluctuates with hydration levels. The hydration sensor could be a bioimpedance spectroscopy sensor to measure impedance or a galvanic skin response (GSR) sensor configured to detect changes in electrical conductance on the wearer's skin surface indicative of sweat production. The hydration sensor can be used to infer sweat rate and provide continuous, real-time measurement of sweat rate, hydration status, and electrolyte balance. Any suitable hydration sensor may be used, including but not limited to the Analog Devices AD5933 impedance sensor.

The processing unit 380 of the computing device 300 is configured to either receive hydration data from the hydration sensor or determine hydration data indicative or representative of the hydration level or water content of the wearer's body. In one embodiment, there are a plurality of hydration sensors positioned at anatomically strategic locations in the garment 100, such as at the lumbar region, thorax, and underarms of the wearer. The processing unit 380 can be configured to analyse or monitor the hydration data to prevent dehydration, heatstroke, or performance loss, and assess fluid losses during prolonged exercise, enhancing physical endurance and reducing the risk of heat-related issues or injuries. Declining hydration levels over time, especially if they fall below a threshold, can indicate fatigue and an increased likelihood of injury due to reduced motor control and reaction times. The hydration data or information from its analysis can be transmitted, for example, to the remote electronic device 700. For example, an alert that the wearer is at an increased risk of injury or fatigue due to dehydration can be sent if their hydration level is continuously below a predetermined threshold value for a set period or their fluid loss is continuously above a predetermined threshold value for a set period. Such an alert can trigger recovery interventions.

### Temperature Sensor

The temperature sensor comprises at least one infrared or thermocouple sensing element to detect the temperature on the wearer's skin, preferably at one or more near-core temperature sites (e.g., the wearer's armpits). The human body maintains an optimal internal or core temperature through thermoregulation. During physical exertion, core temperature rises, and the body initiates sweating and increasing blood flow to the skin to cool down. Continuous temperature monitoring of the wearer helps detect signs of overheating, dehydration or hypothermia.

The processing unit 380 of the computing device 300 is configured to either receive temperature data from the temperature sensor or determine temperature data indicative or representative of the core temperature of the wearer's body. The processing unit 380 can be configured to analyse or monitor the temperature data to prevent heat or cold-related illnesses, optimise training and recovery by monitoring temperature changes, and enhance the wearer's athletic performance in environments with extreme temperatures.

Changes in core temperature levels over time, especially if they fall above or below a threshold, can indicate fatigue, heat stress, or dehydration. The temperature data or information from its analysis can be transmitted, for example, to the remote electronic device 700. For example, an alert that the wearer is at an increased risk of injury or fatigue due to heat stress or dehydration can be sent if their core temperature level is continuously above or below predetermined threshold values for a set period.

### VO₂ Max Sensor

The VO₂ max sensor is configured to estimate the VO₂ max value or maximal oxygen uptake by the wearer as an indicator of their aerobic capacity. VO₂ max reflects the body's maximum ability to utilise oxygen during intense exercise and is closely related to cardiovascular and respiratory efficiency. Traditionally, VO₂ max testing requires gas exchange analysis. However, the sensor estimates it based on the wearer's heart rate (e.g., by multiplying the ratio of maximum heart rate to resting heart rate with a conversion factor) and/or activity intensity. Any suitable VO₂ max sensor may be used. Rather than use a separate VO₂ max sensor, the VO₂ max sensor may comprise one or more of the abovementioned sensors that measure the wearer's heart rate and/or activity intensity.

The processing unit 380 of the computing device 300 is configured to either receive VO₂ max data from the VO₂ max sensor or determine VO₂ max data indicative or representative of the VO₂ max level of the wearer's body. The processing unit 380 can be configured to analyse the VO₂ max data to assess the wearer's cardiovascular fitness and endurance capacity, which are valuable in aerobic conditioning programs. A low VO₂ max level indicates poor cardiovascular fitness and a greater fatigue risk. The VO₂ max data or information from its analysis can be transmitted, for example, to the remote electronic device 700. For example, an alert that the wearer is at an increased risk of injury or fatigue can be sent if their estimated VO₂ max level is continuously below a predetermined threshold value for a set period.

### Processing Unit

The processing unit 380 of the computing device 300 is configured to analyse the biometric or physiological data received from the sensors 200-290. This analysis can comprise values or average values from one or more of the sensors 200-290. For example, a workload sustainability index (WSI) score that assesses how much load the wearer can sustain before becoming fatigued can be calculated by the processing unit 380 using multiple sensor values that comprise force metrics (measured via accelerometers and strain sensors) and cardiovascular effort data (heart rate, VO₂ max, and heart rate variability) as follows:

$$WSI = \frac{{IL}_{avg} + {CL}_{avg}}{{HRV}_{norm} + VO {2}_{max}}$$

where IL_{avg} is the wearer's average impact load (G-force intensity and cumulative hits), CL_{avg} is the wearer's average cardio load (heart rate response and blood oxygen saturation levels), HRVₙₒᵣₘ is the wearer's heart rate variability (HRV) normalised to a baseline (indicator of recovery and fatigue), and VO2ₘₐₓ is the wearer's estimated VO₂ max value (aerobic efficiency and endurance). The wearer's WSI score is used to proactively manage training loads, adjust in-game tactics, and monitor fatigue thresholds. If the wearer's WSI score is less than 1.5, the wearer is at low risk of fatigue and related injury. If the wearer's WSI score is between 1.5 to 3.0, the wearer has a moderate risk of fatigue and their workload should be monitored. If the wearer's WSI is greater than 3.0, the wearer is at high risk of fatigue and related injury and immediate recovery is needed. The processing unit 380 may be configured to send an alert to the remote electronic device 700 that the wearer is at an increased risk of fatigue and related injury if the wearer's WSI score is continuously above 3.0 for a set period.

The processing unit 380 of the computing device 300 is configured by supervised or unsupervised machine learning algorithms (such as, random forest classifiers, gradient boosting trees, and/or convolutional neural networks) stored in its memory 390 to process data from the sensors 200-290 in real time to generate or identify predictive insights, trends, post-game or training summaries, and actionable recommendations on the wearer's training, recovery and/or physical health, for example, “reduce training intensity for Player A to prevent overtraining” or “switch Player B to defence; fatigue level high for current offensive role”. The models may be trained on historical athlete datasets to compute fatigue risk scores, recovery need indices, and impact/cardio load (ICL) ratios. These outputs may be used to make adaptive training recommendations, real-time substitution decisions, and injury-risk alerts. The algorithms may be sport or role specific, for example, to generate insights into a wearer's tackle effectiveness in rugby or a wearer's positioning in football. The processing unit 380 of the computing device 300 may use the algorithms to continuously refine fatigue and injury risk models based on historical and real-time sensor data and to identify wearer-specific risk thresholds for injury prevention.

The processing unit 380 of the computing device 300 may also be configured to correlate a wearer's impact forces, biometric responses, and fatigue levels to generate predictive workload insights for the wearer. A decrease in hydration levels or rising HRV of a wearer may result in a recommendation that the wearer be replaced, substituted or rested based on their fatigue or injury risk. This data or information from its analysis can be automatically transmitted in real-time to a remote electronic device 700, for example, to be viewed on a display by a coach or medical professional or made available to another user, such as a television broadcaster. A graph or chart produced from the data analysis may be displayed to the coach, medical professional, or other user, for example, a bar chart showing the cumulative impacts on a wearer during a game or training session against the g-force of those impacts.

Some or all of the sensor data or information from its analysis may be broadcast in real-time during a live sporting event, such as a televised rugby game. If the wearer is part of a team or military unit comprising one or more other garment wearers, the same remote electronic device 700 and display may be conveniently used to receive and display data and information from multiple wearers in the team or unit, for example, a dashboard showing the WSI score of each wearer.

### Protective Zones

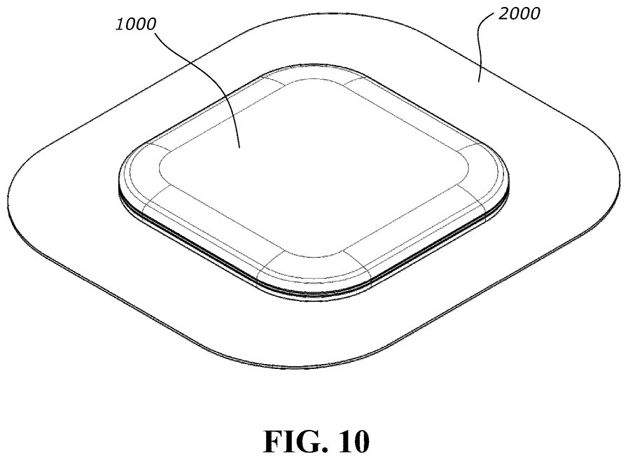

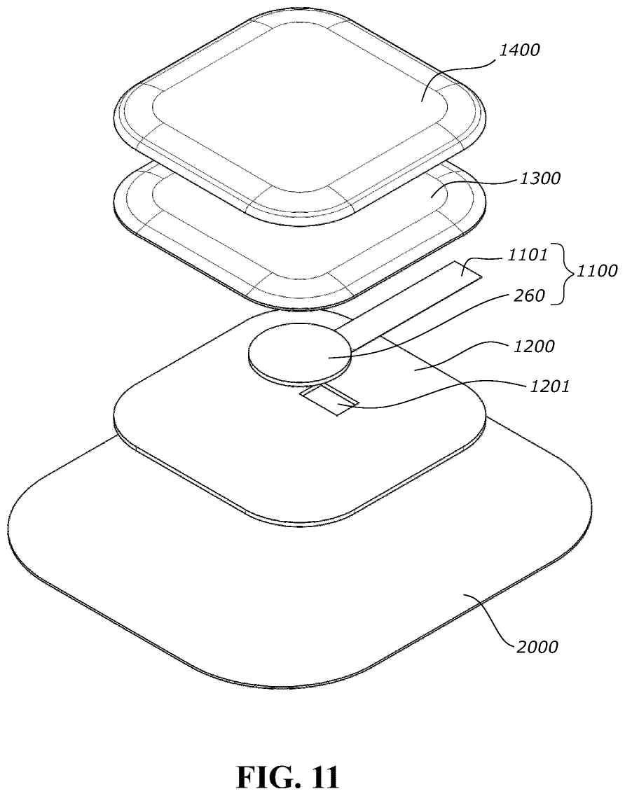

FIGS. 10 to 12 show a first embodiment of a protective zone 1000 located on the fabric layer 2000, for example, on a wearer's thigh 180 or any other portion of their body. As shown in FIG. 11, the protective zone 1000 comprises a sensor layer 1100 interposed between a first or inner impact-absorbing pad 1200 and a second or outer impact-absorbing pad 1300. An optional rigid or semi-rigid protective layer 1400 may be positioned over the second impact-absorbing pad 1300 as the outermost layer. In the exemplified embodiment, the first 1200 and second 1300 impact-absorbing pads and the protective layer 1400 are generally rectangular with rounded corners. However, it will be appreciated that these pads and the protective layer could be any suitable shape.

The first impact-absorbing pad 1200 is a soft cushioning layer abutting the fabric layer 2000 to enhance wearer comfort and distribute localised pressure. The first impact-absorbing pad 1200 serves as padding between the fabric layer 2000 on the wearer's body and the embedded electronic components of the sensor layer 1100. The sensor layer 1100 comprises a sensor, such as an impact sensor 260 on the wearer's thigh 180, and an electrical conduit 1101 operably connected at one end region 1103 to the sensor and at the other end region 1102 to the computing device 300, possibly via another sensor. The first impact-absorbing pad 1200 may contain an aperture 1201 to receive the end region 1103. The electrical conduit 1101 may be implemented as a printed flexible circuit, conductive yarn, or similar high-durability technology on, in, or under the fabric layer 2000 to be routed through the garment 100. The electrical conduit 1101 could comprise one or more wires, fibre optic cables, silicon-based flexible circuits, or flexible printed circuit boards (PCBs), to provide robust and reliable data transmission under dynamic conditions.

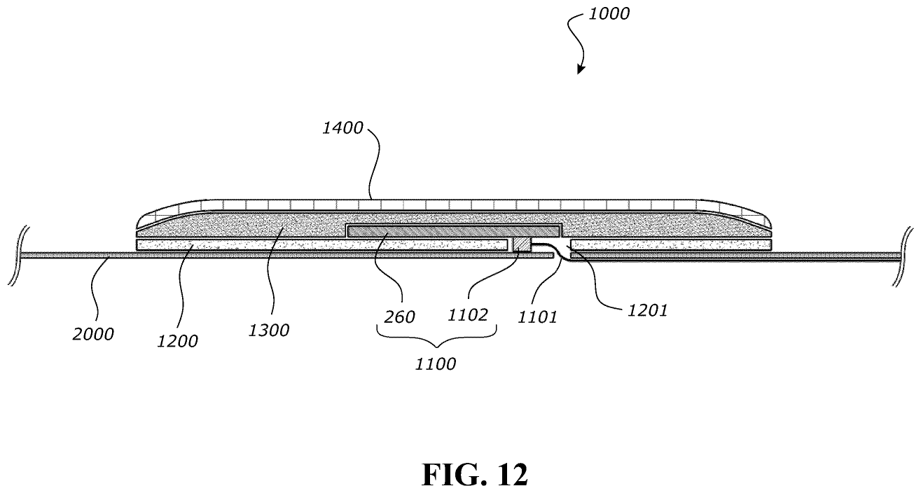

As shown in FIG. 12, the second impact-absorbing pad 1300 is the primary padding layer as it is substantially thicker than the first impact-absorbing pad 1200, and provides impact protection while maintaining the garment's flexibility and user comfort. The first 1200 and second 1300 impact-absorbing pads are capable of flexing with the wearer's movement may be made of any suitable foam pad material, such as ethylene-vinyl acetate (EVA). In one embodiment, the first impact-absorbing pad 1200 is a low-density foam pad, and the second impact-absorbing pad 1300 is a high-density foam pad.

The optional protective layer 1400 is a rigid or semi-rigid layer covering the second impact-absorbing pad 1300 for environments requiring enhanced protection. The protective layer 1400 may comprise any suitably robust or abrasive resistant material, such as polycarbonate, thermoplastic polyurethane (TPU), ultra-high molecular weight polyethylene (UHMWPE), glass fibre reinforced polymer (GFRP), carbon fibre composites, or aramid fibre composites (e.g., Kevlar®), chosen based on specific mission or sporting requirements for impact resistance and weight optimisation. The protective layer 1400 may be configured to be removed from the rest of the protective zone 1000 for laundering in multi-use garments 100.

In the embodiment shown in FIGS. 10 to 12, the second impact-absorbing pad 1300 and the protective layer 1400 are substantially flat with a bevelled or chamfered edge to give the protective zone 1000 a substantially flat outer surface.

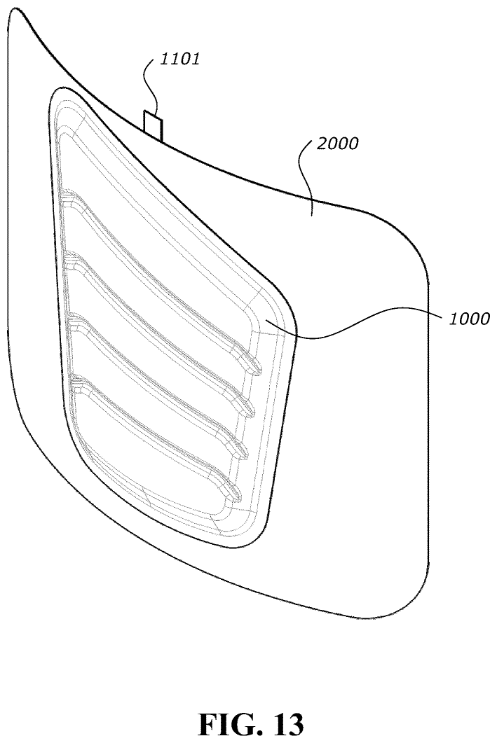

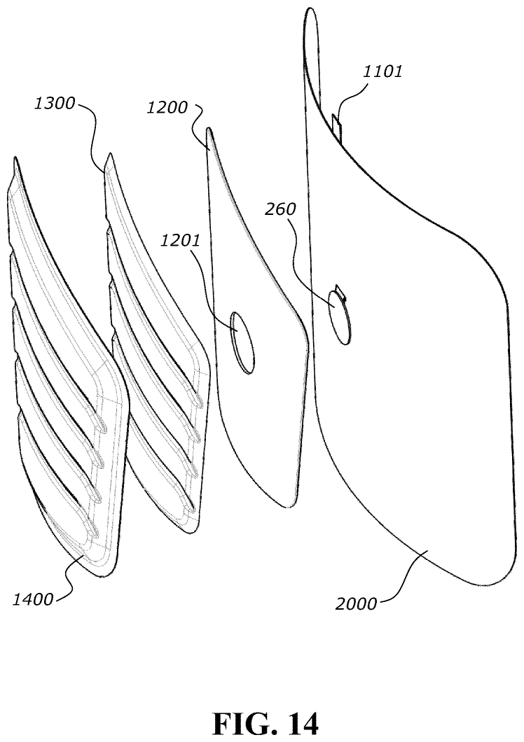

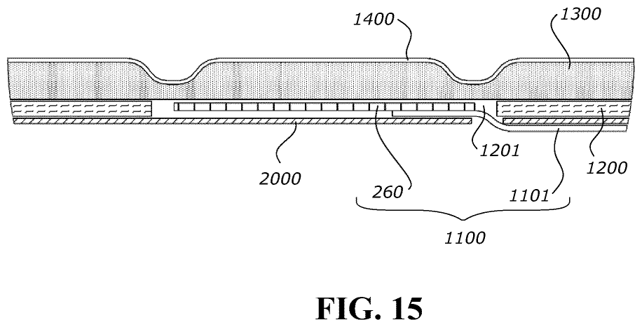

FIGS. 13 to 15 show a second embodiment of a protective zone 1000 located on a section of the fabric layer 2000. The protective zone 1000 of the second embodiment is generally similar to that of the first embodiment described above, and the same reference numbers will be used to refer to the same features. However, unlike in the first embodiment, the aperture 1201 in the first impact-absorbing pad 1200 is sized and shaped to receive the sensor 260. Additionally, rather than being substantially flat, the second impact-absorbing pad 1300 and the protective layer 1400 each contain a plurality of spaced parallel grooves that, as shown in FIG. 15, are configured to engage with each other.

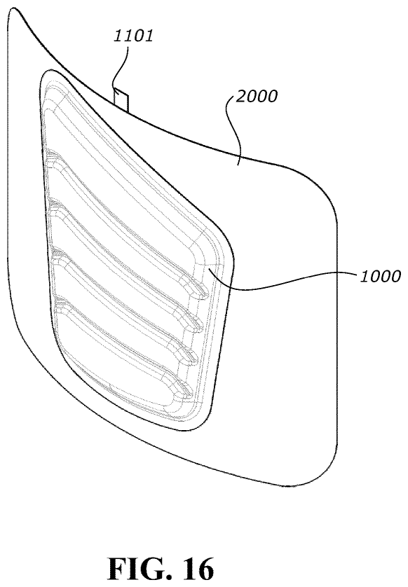

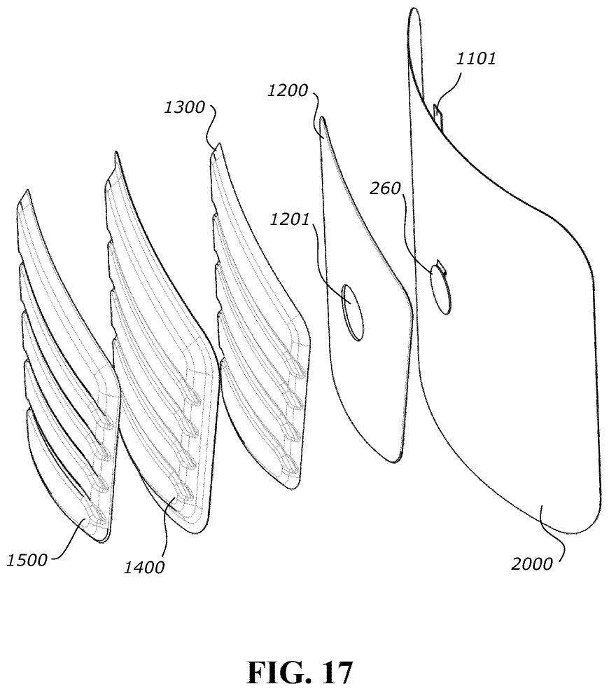

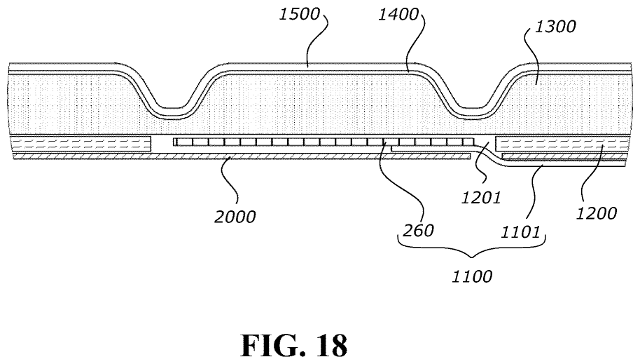

FIGS. 16 to 18 show a third embodiment of a protective zone 1000 located on a section of the fabric layer 2000. The protective zone 1000 of the third embodiment is generally similar to that of the second embodiment described above, and the same reference numbers will be used to refer to the same features. However, unlike the second embodiment, the protective zone 1000 includes an armour layer 1500 over the protective AI layer 1400. In the third embodiment, the armour layer 1400 is a single exoskeleton of any suitable bulletproof material (e.g., Kevlar®) that entirely covers the protective layer 1400 for protecting the wearer in military or high-threat tactical environments, such as an armed conflict or fighting during a war.

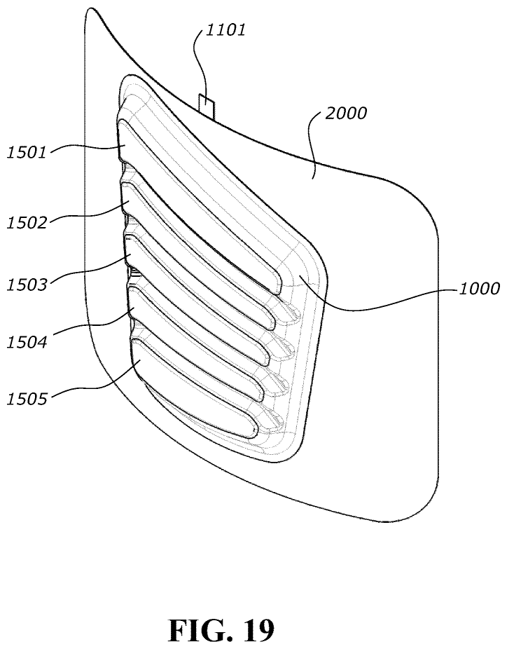

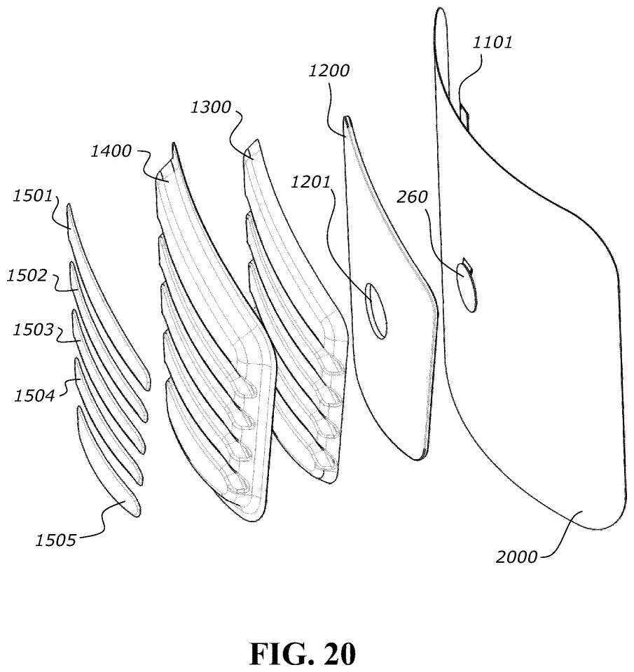

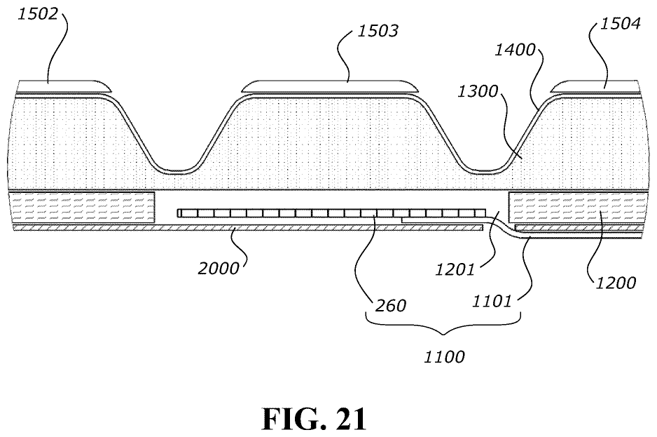

FIGS. 19 to 21 show a fourth embodiment of a protective zone 1000 located on a section of the fabric layer 2000. The protective zone 1000 of the fourth embodiment is generally similar to that of the third embodiment described above, and the same reference numbers will be used to refer to the same features. However, unlike the third embodiment, for greater wearer flexibility, the armour layer comprises a plurality of armoured pieces 1501, 1502, 1503, 1504, 1505, each piece covering a substantially flat portion of the protective layer 1400.

### Data Management

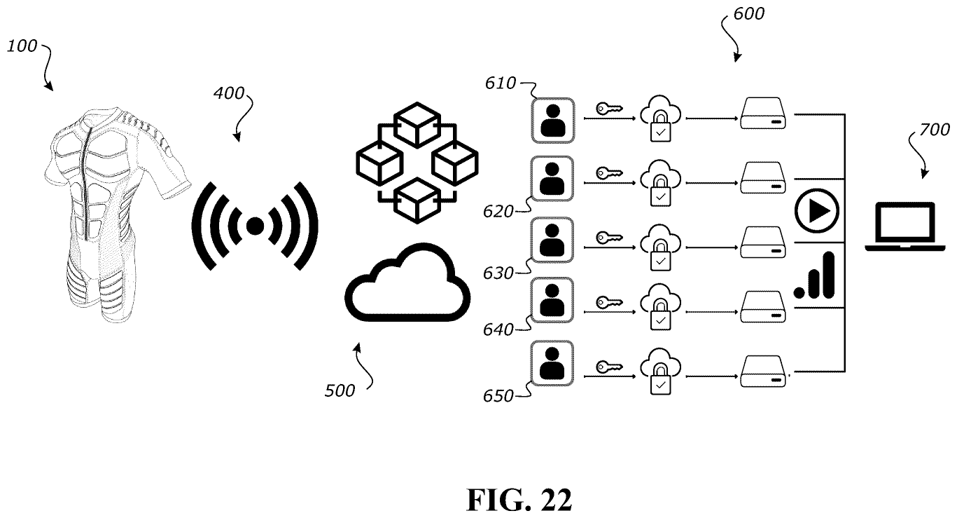

Using the touchscreen 320 or buttons 330 on the computing device 300, each wearer can adjust data management settings to control and share their data and the real-time information generated or identified by its analysis with coaches, medical staff, performance analysts, or other users as needed. Accordingly, the wearer maintains control over their collected biometric or physiological data, for example, using secure cryptographic or blockchain-based data management, such as Hyperledger Fabric or another suitable private blockchain, to determine who can access, store, and analyse the wearer's data. FIG. 22 shows a process for transmitting data from the garment 100 to a remote electronic device 700. At step 400, encrypted data from the sensors 200-290 or encrypted information from its analysis is wirelessly transmitted to the cloud 500 as needed.

Because the computing device 300 handles sensitive biometric data directly on the device, only the necessary data or information needs to be transmitted, protecting wearer privacy and confidential or classified information. This also significantly reduces latency and ensures rapid responses to changes in the wearer's athletic performance. A decentralised cloud data management platform 500 powered by blockchain technology can be used by wearers, with the wearers retaining ownership of their performance and health data, deciding when and how it is shared with coaches, teams, and other stakeholders. This ensures data portability across teams, leagues, or professional organisations, as well as privacy, addressing rising player concerns over athlete data security in professional sports. For example, at step 600, a wearer can decide to give five people 610-650 access to their data, such as a coach, trainer, medical professional, physiotherapist, and manager. In this example, only the five approved people 610-650 can access the wearer's data via a remote electronic device 700. The approved people 610-650 may be given access to the same or different data and receive customised alerts depending upon their role, for example, different data and alerts may be provided to a coach compared to a trainer.

Data from the sensors 200-290 can be stored in the memory 390 of the computing device 300, for example, for post-game or training analysis. The processing unit 380 may be configured to analyse patterns in the stored sensor data to predict potential injury risks to the wearer. For example, employing unsupervised anomaly detection using clustering and principal component analysis (PCA), the processing unit 380 could analyse patterns related to abnormal increases in the wearer's heart rate coupled with low HRV, high cumulative impact levels over time or repeatedly high g-force spikes, dehydration or drops in blood oxygen saturation, and/or low blood glucose levels.

The processing unit 380 can also be configured to analyse the impact of fatigue on injury risks, particularly focusing on examining any correlations between indicators of fatigue (e.g., elevated heart rate, reduced heart rate variability, or declining hydration levels) and injury-prone metrics like cumulative impact levels or abrupt changes in motion. For example, the processing unit 380 could identify fatigue indicators (e.g., heart rate trends, HRV, hydration levels, and blood oxygen saturation), measure correlations with injury-prone metrics (e.g., g-force impact levels or abrupt motions), and forecast trends over time using recurrent neural networks (RNNs), such as long short-term memory (LSTM) architectures. These trends could include analysing cumulative fatigue effects to examine whether wearers who show prolonged periods of fatigue indicators are experiencing more frequent or severe impacts. Additionally, the trends could include examining recovery cycles to analyse periods with reduced fatigue indicators to understand if recovery lowers injury risk. A correlation and time-series trend analysis may be run to assess if there is a relationship between the fatigue indicators and impact-related metrics in the stored sensor data.

The garment 100 represents a significant advancement in wearable sports technology by integrating protection, performance monitoring, and recovery support into a single garment designed for athletes in contact sports or military personnel during training or deployment. The invention not only enhances the safety and performance of wearers but also gives them control over their biometric data, addressing key concerns with data privacy.

Preferred embodiments of the invention have been described by way of example only, and modifications may be made thereto without departing from the scope of the invention.

1. An exercise or sporting garment for a wearer, the garment comprising:
a) a carrier arranged to removably carry a computing device;
b) one or more sensors integrated into the garment and configured to sense and send biometric or physiological data about the wearer to the computing device; and
c) one or more protective zones, the or each protective zone of the garment comprising a plurality of overlaid pads arranged to protect a region of the wearer's body and flex with the wearer's movement;
wherein the computing device is configured to analyse the biometric or physiological data received from at least one of the sensors and transmit the data or information derived at least in part from the analysis of the data to a remote electronic device.

2. The garment according to claim 1, wherein the computing device is configured to analyse the biometric or physiological data received from at least one of the sensors and transmit the data or information in real-time.

3. The garment according to claim 1, wherein the garment is configured to dynamically adjust one or more characteristics of the or each protective zone.

4. The garment according to claim 1, wherein the or each protective zone comprises a sensor interposed between an inner impact-absorbing pad and an outer impact-absorbing pad.

5. The garment according to claim 4, wherein the garment comprises a fabric layer, the inner impact-absorbing pad of the or each protective zone abutting the fabric layer, and the outer impact-absorbing pad of the or each protective zone being substantially thicker than the inner impact-absorbing pad.

6. The garment according to claim 4, wherein the or each protective zone comprises a rigid or semi-rigid protective layer covering or partially covering the outer impact-absorbing pad.

7. The garment according to claim 6, wherein the or each protective zone comprises an armour layer covering or partially covering the protective layer.

8. The garment according to claim 1, wherein the or each protective zone is arranged to protect a region of the wearer's body selected from the wearer's shoulder, chest, abdomen, flank, back, and thigh.

9. The garment according to claim 1, wherein the garment contains one or more electrical conduits, electrical connection networks, or data bus having a first end region operably connected to the carrier or the computing device and a second end region operably connected to the or at least one sensor.

10. The garment according to claim 1, wherein the carrier is a docking structure adapted to releasably receive the computing device.

11. The garment according to claim 1, wherein the or each sensor comprises a sensor selected from an ECG sensor, a PPG sensor, a blood oxygen saturation sensor, a blood glucose sensor, a blood lactate sensor, a GPS sensor, a g-force or cumulative workload sensor, a hydration sensor, a temperature sensor, and a VO₂ max sensor.

12. A computing device arranged to be carried in a carrier on an exercise or sporting garment for a wearer, the garment comprising:
a) one or more sensors integrated into the garment and configured to sense and send biometric or physiological data about the wearer to the computing device; and
b) one or more protective zones, the or each protective zone of the garment comprising a plurality of overlaid pads arranged to protect a region of the wearer's body and flex with the wearer's movement;
wherein a processing unit of the computing device is configured to analyse the biometric or physiological data received from at least one of the sensors and transmit the data or information derived at least in part from the analysis of the data to a remote electronic device.

13. The computing device according to claim 12, wherein the processing unit is configured to transmit the biometric or physiological data or information derived at least in part from the analysis of the data to a remote electronic device in real time.

14. The computing device according to claim 12, wherein the computing device is configured to generate predictive workload insights for the wearer at least in part from the biometric or physiological data and transmit this information to the remote electronic device.

15. The computing device according to claim 12, wherein the computing device is configured to assess cumulative fatigue or injury risk for the wearer at least in part from the biometric or physiological data and transmit this information to the remote electronic device.

16. The computing device according to claim 12, wherein the computing device is configured to identify anomalous impact patterns or physiological stress responses at least in part from the biometric or physiological data and transmit this information to the remote electronic device.

17. A method of analysing biometric or physiological data about a wearer of an exercise or sporting garment, the method using a processing unit of the computing device configured to implement the steps of:
a) receiving the data from one or more sensors integrated into the garment;
b) analysing the data to derive information at least in part from the analysis of the data; and
c) transmitting the data or the information derived from the data to a remote electronic device;
wherein the garment comprises a carrier arranged to removably carry the computing device and one or more protective zones, the or each protective zone comprising a plurality of overlaid pads arranged to protect a region of the wearer's body and flex with the wearer's movement.

18. The method according to claim 17, further comprising the step of sending an alert to the remote electronic device that the wearer is at an increased risk of injury or fatigue based at least in part on the analysis of the biometric or physiological data.

19. The method according to claim 17, wherein the biometric or physiological data comprises any one or more of the following: cardiac data indicative or representative of the health or condition of the wearer's heart; blood oxygen data indicative or representative of the heart rate or blood oxygen level of the wearer; blood volume data indicative or representative of the blood oxygen level of the wearer; blood glucose data indicative or representative of the blood glucose level of the wearer; blood lactate data indicative or representative of the blood lactate concentration of the wearer; GPS data indicative or representative of the location or movement of the wearer; g-force or impact intensity data indicative or representative of the acceleration force on the wearer's body; hydration data indicative or representative of the hydration level or water content of the wearer's body; temperature data indicative or representative of the core temperature of the wearer's body; and VO₂ max data indicative or representative of the VO₂ max level of the wearer's body.

20. An electronically-implemented method comprising software code or coded instructions that are executable or implemented by a computer processor, or controller to carry out the method of claim 17.
